(19) United States
(12) Patent Application Publication
(10) Pub. No.: US 2026/0245705 A1
(43) Pub. Date: Aug. 20, 2026

(54) METHOD AND APPARATUS FOR DYNAMIC REFINEMENT AND WEIGHTED AGGREGATION OF MULTIPLE INFERENCES

(71) Applicant: Nokia Solutions and Networks Oy, Espoo (FI)

(72) Inventors: Aidan BOYD, Murray Hill, NJ (US); Huseyin UZUNALIOGLU, Westford, MA (US); Md Motiur RAHMAN, Murray Hill, NJ (US)

(21) Appl. No.: 19/424,671

(22) Filed: Dec. 18, 2025

(30) Foreign Application Priority Data
Feb. 19, 2025 (FI) 20255146

(51) Int. Cl. G16H 30/40; G06N 3/0464; G06N 3/08; G06T 5/70; G06T 7/10; G16H 50/20

(52) U.S. Cl. CPC G16H 30/40; G06N 3/0464; G06N 3/08; G06T 5/70; G06T 7/10; G16H 50/20; G06T 2207/20182

(57) ABSTRACT

A method for generating a unified prediction includes generating a plurality of predictions of an input based on a plurality of local prediction models, determining a confidence level of the plurality of predictions, assigning weights to the plurality of predictions based on the respective confidence levels, and generating the unified prediction based on the plurality of predictions and the respective weights.

### BACKGROUND

In many industries, organizations are hesitant or unable to share proprietary data with each other due to privacy concerns and competitive pressures, making access to diverse and extensive datasets challenging. This reluctance limits the availability of varied data for training effective artificial intelligence (AI) models and leads to significant variations in model quality and characteristics across different institutions.

### SUMMARY

The scope of protection sought for various example embodiments of the disclosure is set out by the independent claims. The example embodiments and/or features, if any, described in this specification that do not fall under the scope of the independent claims are to be interpreted as examples useful for understanding various embodiments.

At least one example embodiment provides a method for generating a unified prediction, the method including generating a plurality of predictions of an input based on a plurality of local prediction models, determining a confidence level of the plurality of predictions, assigning weights to the plurality of predictions based on the respective confidence levels, and generating the unified prediction based on the plurality of predictions and the respective weights.

According to at least one example embodiment, the plurality of local prediction models may be trained for image segmentation, and the generating the unified prediction includes generating an image segmentation of the input.

According to at least one example embodiment, the input may be at least one of a medical image or a medical image dataset, and the generating the unified prediction includes generating an image segmentation map.

According to at least one example embodiment, each prediction model of the plurality of prediction models may correspond with a different respective source of a plurality of sources.

According to at least one example embodiment, each local prediction model is trained by the respective different source.

According to at least one example embodiment, the method further includes receiving the plurality of local prediction models from the different respective sources.

According to at least one example embodiment, the method further includes denoising the plurality of predictions by adjusting predictions differing from an average of the plurality of predictions by greater than a threshold.

According to at least one example embodiment, the denoising includes replacing a failed prediction with the average of the plurality of predictions.

According to at least one example embodiment, the determining the confidence level includes determining an importance of each prediction, and evaluating an amount of meaningful information of each prediction.

According to at least one example embodiment, the evaluating the amount of meaningful information of each prediction includes evaluating the amount of meaningful information of each prediction based on an average pooling of the prediction and a maximum pooling of the prediction.

According to at least one example embodiment, the method further includes training a general prediction model by the determining, the assigning, and the generating.

According to at least one example embodiment, the general prediction model includes the plurality of local prediction models.

According to at least one example embodiment, the generating the unified prediction includes generating the unified prediction using a convolutional network based on the plurality of predictions and the respective weights.

At least one example embodiment provides an apparatus including at least one memory storing instructions and at least one processor configured to execute the instructions and cause the device to generate a plurality of predictions of an input based on a plurality of local prediction models, determine a confidence level of the plurality of predictions, assign weights to the plurality of predictions based on the respective confidence levels, and generate a unified prediction based on the plurality of predictions and the respective weights.

According to at least one example embodiment, the plurality of local prediction models may be trained for image segmentation, and the at least one processor is configured to execute the instructions to cause the apparatus to generate the unified prediction by generating a prediction of at least one malady based on the medical image or the medical image dataset.

According to at least one example embodiment, the input may be at least one of a medical image or a medical image dataset, and the at least one processor is configured to execute the instructions to cause the apparatus to generate the unified prediction by generating an image segmentation map.

According to at least one example embodiment, each prediction model of the plurality of prediction models corresponds with a different respective source of a plurality of sources.

According to at least one example embodiment, each local prediction model is trained by the respective different source.

According to at least one example embodiment, the at least one processor is configured to execute the instructions to cause the apparatus to receive the plurality of local prediction models from the different respective sources.

According to at least one example embodiment, the at least one processor is configured to execute the instructions to cause the apparatus to denoise the plurality of predictions by adjusting predictions differing from an average of the plurality of predictions by greater than a threshold.

According to at least one example embodiment, the at least one processor is configured to execute the instructions to cause the apparatus to denoise the plurality of predictions by replacing a failed prediction with the average of the plurality of predictions.

According to at least one example embodiment, the at least one processor is configured to execute the instructions to cause the apparatus to determine the confidence level by determining an importance of each prediction, and evaluating an amount of meaningful information of each prediction.

According to at least one example embodiment, the at least one processor is configured to execute the instructions to cause the apparatus to evaluate the amount of meaningful information of each prediction based on an average pooling of the prediction and a maximum pooling of the prediction.

According to at least one example embodiment, the at least one processor is configured to execute the instructions to cause the apparatus to train a general prediction model by determining the confidence level of the plurality of predictions, assigning the weights to the plurality of predictions, and generating the unified prediction.

According to at least one example embodiment, wherein the trained general prediction model includes the plurality of local prediction models.

According to at least one example embodiment, the at least one processor is configured to execute the instructions to cause the apparatus to generate the unified prediction using a convolutional network based on the plurality of predictions and the respective weights.

At least one example embodiment provides a non-transitory computer-readable storage medium storing computer-readable instructions that, when executed, cause one or more processors to cause an apparatus to perform a method for generating a unified prediction, the method including generating a plurality of predictions of an input based on a plurality of local prediction models, determining a confidence level of the plurality of predictions, assigning weights to the plurality of predictions based on the respective confidence levels, and generating the unified prediction based on the plurality of predictions and the respective weights.

At least one example embodiment provides a device including a means for generating a plurality of predictions of an input based on a plurality of local prediction models, a means for determining a confidence level of the plurality of predictions, a means for assigning weights to the plurality of predictions based on the respective confidence levels, and a means for generating a unified prediction based on the plurality of predictions and the respective weights.

### BRIEF DESCRIPTION OF THE DRAWINGS

Example embodiments will become more fully understood from the detailed description given herein below and the accompanying drawings, wherein like elements are represented by like reference numerals, which are given by way of illustration only and thus are not limiting of this disclosure.

FIG. 1 is a block diagram illustrating an example system according to example embodiments.

FIG. 2 is a block diagram illustrating an example apparatus according to example embodiments.

FIG. 3 is a logical illustration of a personalized mixture of local experts (P-MoLE) model according to example embodiments.

FIG. 4 illustrates an example of noise in predictions of individual models.

FIG. 5 shows an example algorithm for generating a channel-wise weighted prediction map.

FIG. 6 is a flowchart illustrating a method for training a P-MoLE model according to example embodiments.

FIG. 7 is a flowchart illustrating a method for generating a prediction using a P-MoLE model according to example embodiments.

It should be noted that these figures are intended to illustrate the general characteristics of methods, structure and/or materials utilized in certain example embodiments and to supplement the written description provided below. These drawings are not, however, to scale and may not precisely reflect the precise structural or performance characteristics of any given embodiment, and should not be interpreted as defining or limiting the range of values or properties encompassed by example embodiments. The use of similar or identical reference numbers in the various drawings is intended to indicate the presence of a similar or identical element or feature.

### DETAILED DESCRIPTION

Various example embodiments will now be described more fully with reference to the accompanying drawings in which some example embodiments are shown.

Detailed illustrative embodiments are disclosed herein. However, specific structural and functional details disclosed herein are merely representative for purposes of describing example embodiments. The example embodiments may, however, be embodied in many alternate forms and should not be construed as limited to only the embodiments set forth herein.

It should be understood that there is no intent to limit example embodiments to the particular forms disclosed. On the contrary, example embodiments are to cover all modifications, equivalents, and alternatives falling within the scope of this disclosure. Like numbers refer to like elements throughout the description of the figures.

Although the specification may refer to “an”, “one”, or “some” embodiment(s) in several locations of the text, this does not necessarily mean that each reference is made to the same embodiment(s), or that a particular feature only applies to a single embodiment. Single features of different embodiments may also be combined to provide other embodiments. Further, when a particular feature, structure, or characteristic is described in connection of an embodiment, it is within the knowledge of one skilled in the art to apply such feature, structure, or characteristic in connection with other embodiments whether or not explicitly de-scribed. It shall be understood that although the terms “first,” “second” and the like may be used herein to describe various elements, these elements should not be limited by these terms. These terms are only used to distinguish one element from another.

For the purposes of the present disclosure, the phrases “at least one of A or B”, “at least one of A and B”, and “A and/or B” means (A), (B), or (A and B). For the purposes of the present disclosure, the phrase “A, B, and/or C” means (A), (B), (C), (A and B), (A and C), (B and C), or (A, B, and C).

Combining multiple inferences from diverse sources has gained traction as a method to enhance prediction accuracy and robustness across various applications. This approach allows the aggregation of information from multiple origins without the need to centralize data, thereby preserving data privacy and integrity. Recently, it has become evident that personalization is crucial, as traditional aggregation methods often struggle to accommodate data heterogeneity and varying data distributions.

Traditional collaborative approaches, such as federated learning (FL), enable organizations to train models collectively without sharing raw data by only exchanging model updates. While federated learning maintains data privacy, a single global model often fails to effectively capture the diversity of data sources, leading to diminished performance and reduced robustness.

To address these limitations, personalized federated learning (PFL) has been developed to create individualized models tailored to each organization's specific data characteristics. Although PFL enhances performance by adapting models to local data, it requires complex synchronization and substantial computational resources, increasing operational costs and coordination efforts.

Some example embodiments overcome these challenges by allowing organizations to independently train models on their private data and share these models to form an ensemble of local expert models LE. By enabling organizations to collaboratively enhance model performance through model sharing rather than data sharing, some example embodiments may maintain data privacy and reduce the overhead associated with traditional PFL.

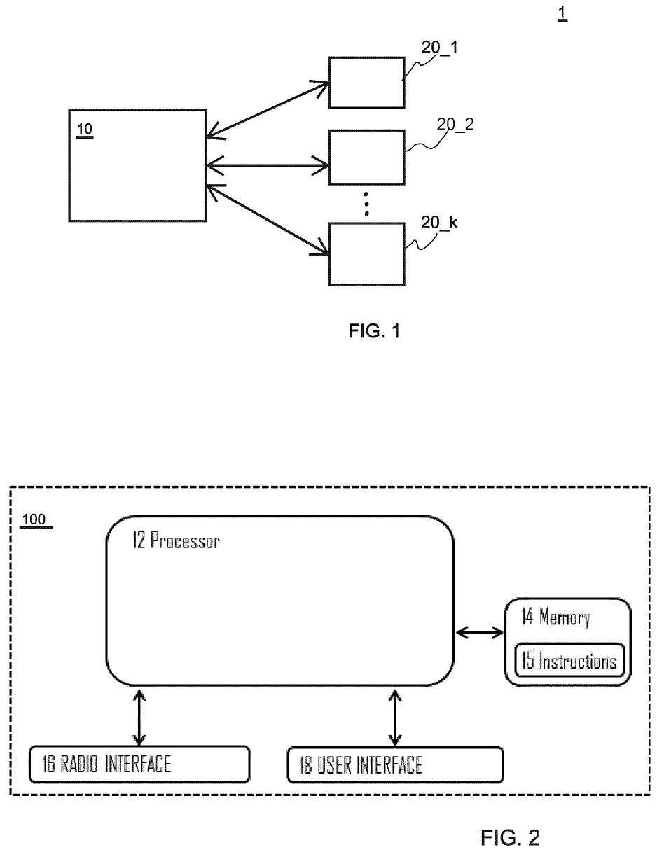

FIG. 1 is an example of a system according to example embodiments.

Referring to FIG. 1, a system 1 may include a server 10 and/or a plurality of terminal devices 20. The plurality of terminal devices 20 may include terminal devices 20_1 to 20_K. A number K of the terminal devices 20 is not particularly limited.

FIG. 2 shows, by way of example, a block diagram of an apparatus 100. The apparatus 100 comprises, for example, at least one processor 12 and at least one memory 14 storing instructions 15 that, when executed by the at least one processor, cause the apparatus 100 at least to perform the method or methods as disclosed herein, and any of the embodiments thereof. In an example, the at least one memory and the instructions (e.g. a computer program code, software), are configured, with the at least one processor, to cause the apparatus 100 to perform the method or methods as disclosed herein, and any of the embodiments there-of.

A processor 12 may comprise circuitry, or be constituted as circuitry or circuitries, the circuitry or circuitries being configured to perform phases of methods in accordance with example embodiments described herein. As used in this application, the term “circuitry” may refer to one or more or all of the following: (a) hardware-only circuit implementations, such as implementations in only analog and/or digital circuitry, and (b) combinations of hardware circuits and software, such as, as applicable: (i) a combination of analog and/or digital hardware circuit(s) with software/firmware and (ii) any portions of hardware processor(s) with software (including digital signal processor(s)), software, and memory (ies) that work together to cause an apparatus, such as a user equipment, to perform various functions) and (c) hardware circuit(s) and or processor(s), such as a microprocessor(s) or a portion of a microprocessor(s), that requires software (e.g., firmware) for operation, but the software may not be present when it is not needed for operation. This definition of circuitry applies to all uses of this term in this application, including in any claims. As a further example, as used in this application, the term circuitry also covers an implementation of merely a hardware circuit or processor (or multiple processors) or portion of a hardware circuit or processor and its (or their) accompanying software and/or firmware. The term circuitry also covers, for example and if applicable to the particular claim element, a baseband integrated circuit or processor integrated circuit for a mobile device or a similar integrated circuit in server, a cellular network device, or other computing or network device.

The memory 14 may be implemented using any suitable data storage technology. The memory may comprise a database for storing data. The memory 14 may be at least in part external to apparatus 100 but accessible to apparatus 100.

The instructions 15 may be comprised in a computer readable medium or a non-transitory computer readable medium. A term non-transitory, as used herein, is a limitation of the medium itself (i.e. tangible, not a signal) as opposed to a limitation on data storage persistency (e.g. random access memory (RAM) vs. read only memory (ROM)).

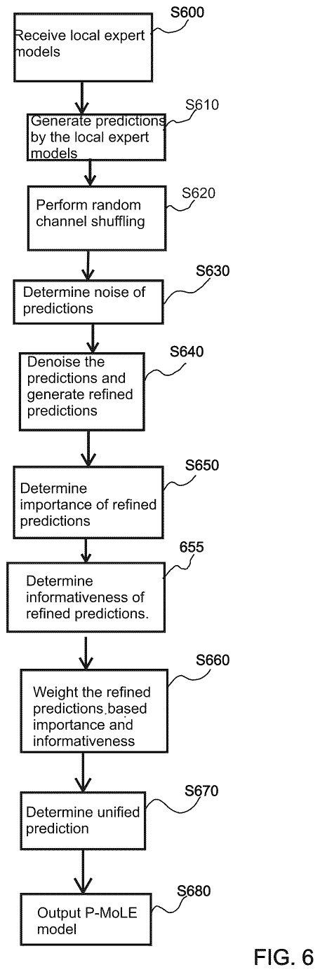

For example, the apparatus 100 is a terminal device, such as the terminal devices 20 of FIG. 1. As another example, the apparatus is comprised in such a terminal device, e.g. as a chipset configured to control the terminal device. The apparatus 100 may be caused or configured to perform at least the method of FIG. 5 and/or FIG. 6 and/or any one or more of the embodiments described.

As another example, the apparatus 100 is a server, e.g. the server 10 of FIG. 1. In another embodiment, the apparatus is comprised in such a server, e.g. as a chipset configured to control the server. The apparatus 100 may be caused or configured to perform at least the method of FIG. 5 and/or FIG. 6 and/or any one or more of the embodiments described.

The apparatus 100 comprises a radio interface 16. The radio interface 16 may provide the apparatus 100 with communication capabilities. The radio interface 16 may comprise a receiver configured to receive information in accordance with at least one cellular or non-cellular standard. The radio interface 16 may comprise a transmitter configured to transmit information in accordance with at least one cellular or non-cellular standard. The receiver may comprise more than one receiver. The transmitter may comprise more than one transmitter. The radio interface 16 may comprise a transceiver configured to receive and transmit information in accordance with at least one cellular or non-cellular standard. The transceiver may comprise more than one transceiver.

The apparatus 100 may comprise a user interface 18 comprising, for example, at least one of a keypad, a microphone, a touch display, a display, a speaker, etc. The user interface 18 may be used to control the apparatus by the user. The user interface 18 may be external to the apparatus 100. For example, the apparatus 100 may be connected to another device, such as a computer, either via wireless or wired connection, and the apparatus 100 is controlled by the user via the computer.

In an embodiment, at least some of the processes described herein may be carried out by an apparatus comprising means for carrying out at least some of the described processes. Means for performing method steps as disclosed herein may include software and/or hardware components of the apparatus 100. For example, the at least one processor 12, the memory 14, and the computer program code form means for carrying out the method or methods as disclosed herein, and any of the embodiments thereof. As used herein the term “means” is to be construed in singular form, i.e. referring to a single element, or in plural form, i.e. referring to a combination of single elements. Therefore, terminology “means for [performing A, B, C]”, is to be interpreted to cover an apparatus in which there is only one means for performing A, B and C, or where there are separate means for performing A, B and C, or partially or fully over-lapping means for performing A, B, C. Further, terminology “means for performing A, means for performing B, means for performing C” is to be interpreted to cover an apparatus in which there is only one means for performing A, B and C, or where there are separate means for performing A, B and C, or partially or fully overlapping means for performing A, B, C.

Referring to FIGS. 1-2, according to some example embodiments, the terminal devices 20 may be, for example, end user devices of individual end users. For example, a terminal devices 20 may be a cellular phone, a tablet computer, a personal computer, etc. In this example, each user independently trains their own local expert model LE using proprietary data that they choose to keep private, sharing only the trained local expert models LE rather than the underlying data.

By collecting and integrating these individual local expert models LE, a personalized mixture of local experts (P-MoLE) model according to example embodiments can deliver enhanced solutions that outperform each standalone local expert model LE. This collaborative approach not only preserves customer data privacy but also harnesses collective insights, resulting in more robust and accurate performance across various applications (e.g., telecom applications). Additionally, the P-MoLE model according to example embodiments leverages advanced security protocols to ensure the integrity and confidentiality of each local expert model LE, enabling seamless and trustworthy collaboration among users. As an example, a network anomaly detection model (e.g., a local expert model LE) can be trained by different telecommunication service providers (SPs) using their respective proprietary data. Rather than sharing their proprietary data, the SPs may share their local expert models LE. Using P-MoLE, each SP can create a personalized mixture of experts model that performs better than their original (own) model.

As another example, different manufacturing factories often encounter similar quality control challenges but cannot share proprietary data due to privacy and competitive concerns. In this example, the terminal devices 20 may be a device and/or server located at a factory site. The P-MoLE model according to example embodiments allows each factory to train its own quality inspection local expert model LE privately and share just this model with the group. The system 1 according to example embodiments securely aggregates and refines inferences from these local expert models LE, allowing factories to benefit from collective expertise without exposing sensitive information. This results in more accurate defect detection and optimized production processes. For example, an anomaly detection model (e.g., a local expert model LE) can be trained by different factories to, for example, detect defective products manufactured in the factory and/or to determine a state of a machine in the factory based on sensor (e.g., vibration, temperature, noise, etc.) measurements. Rather than sharing their proprietary data, the factories may share their local expert models LE. Using P-MOLE, each factory can create a personalized mixture of experts model that performs better than their original (own) model.

As still another example, sharing patient data among hospitals and other healthcare entities is a complex issue, largely governed by privacy laws and regulations like the Health Insurance Portability and Accountability Act (HIPAA) in the United States and the General Data Protection Regulation (GDPR) in the European Union. These regulations are designed to protect patient privacy and ensure that personal health information is handled with care. This means access to medical imaging datasets suitable to train effective AI models is restricted and often times limited in quantity. Additionally, there is significant variation in dataset curation across institutions, such as in the sensors used to capture the data, patient demographics, resolution, annotation protocol, etc. Thus, training robust AI models that can be generalized across patient populations is difficult.

According to some example embodiments, the terminal devices 20 may be devices and/or servers located at medical sites. For example, the medical sites may train their own local expert models LE to, for example, analyze a medical image. The system 1 according to example embodiments securely aggregates and refines inferences from these local expert models LE, allowing medical sites to benefit from collective expertise without exposing sensitive information. This results in more accurate medical image analysis. For example, a medical site may train a local expert model LE for image segmentation. For example, a medical site may train a local expert model LE for polyp detection from colonoscopy images, early detection and diagnosis of ocular pathologies such as diabetic retinopathy, glaucoma, and/or age-related macular degeneration, etc. Rather than sharing their proprietary data (e.g., confidential patient data), the medical sites may share their local expert models LE. Using P-MOLE, each medical site can create a personalized mixture of experts model that performs better than their original (own) model.

For purposes of clarity and conciseness, example embodiments will be mostly discussed with reference to examples corresponding with medical sites training local expert models LE for image analysis. However, example embodiments are not limited to this example.

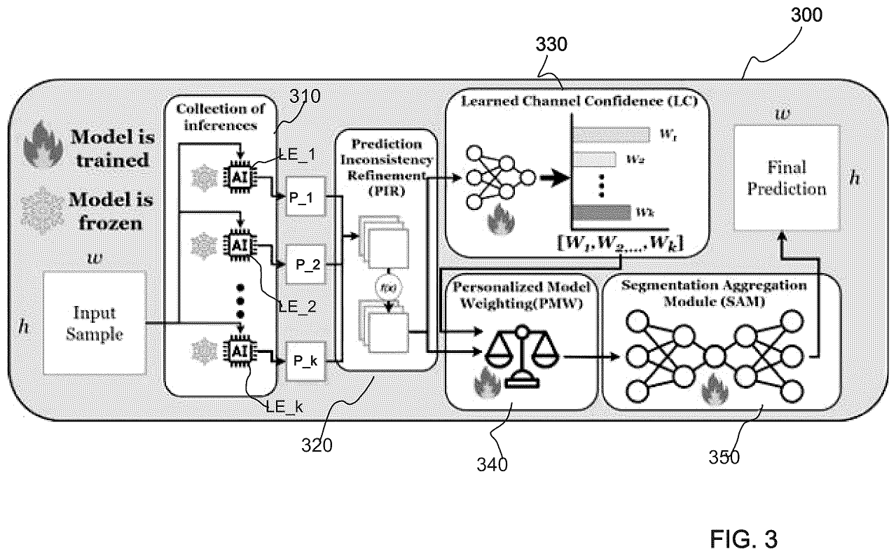

FIG. 3 is a logical illustration of a shared model according to example embodiments.

Referring to FIG. 3, a P-MoLE model 300 according to example embodiments includes a team of experts (ToE) module 310, a prediction inconsistency refinement (PIR) module 320, a learned channel confidence (LC) module 330, a personalized model weighting (PMW) module 340, and/or a segmentation aggregation module (SAM) 350. The P-MoLE model 300 may be referred to as a general prediction model 300.

According to some example embodiments, the P-MoLE model 300 may be trained and/or implemented at the server 10. For example, the P-MoLE model 300 may be trained and/or implemented by the processor 12 of the server 10. However, example embodiments are not limited to this example. For example, according to some example embodiments, the P-MoLE model 300 may be trained and/or implemented at a terminal device 20. For example, the P-MoLE model 300 may be trained and/or implemented by the processor 12 of a terminal device 20.

The ToE module 310 may include a plurality of local expert models LE_1 to LE_K. Each local expert model LE may have been trained by a terminal device 20. For example, each local expert model LE may have been trained at a medical site for image segmentation (e.g., medical image segmentation. For example, each local expert model LE may have been trained for polyp detection from colonoscopy images, early detection and diagnosis of ocular pathologies such as diabetic retinopathy, glaucoma, and/or age-related macular degeneration, etc.

Each local expert model LE included in the ToE module 310 may be frozen. For example, the local expert models LE_1 to LE_K included in the ToE module 310 may no longer be further trained.

A sample input (e.g., sample training data) may be input to the ToE module 310. Each local expert model LE of the ToE 310 may analyze the sample input and generate a respective prediction P_1 to P_K. For example, according to some example embodiments, the sample input may be an image (e.g., a plurality of images, e.g., an image dataset). For clarity and conciseness, the sample input as an image will be mainly discussed. However, example embodiments are not limited to this example.

The PIR module 320 may denoise the predictions P_1 to P_K of the local expert models LE_1 to LE_K.

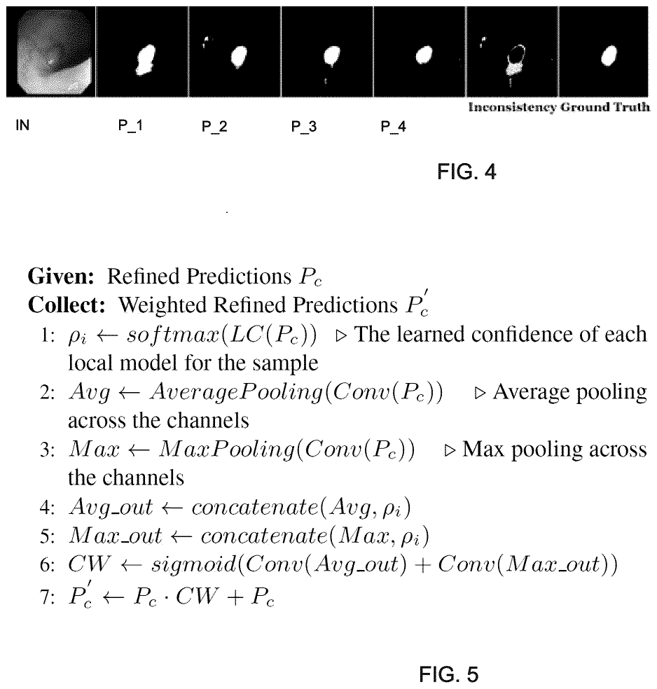

FIG. 4 illustrates an example of inconsistencies in local-model predictions with respect to a ground truth.

FIG. 4 shows an example sample input image IN and four example predictions P_1 to P_4 generated by four respective local expert models LE_1 to LE_4. When multiple independent predictions are made on a sample input IN, the predictions can sometimes disagree due to random errors or true differences in domain understanding of the local expert models LE. To create a more reliable overall result, the PIR module 320 examines these varying predictions and distinguishes between noise (unreliable differences) and meaningful information.

For example, the PIR module 320 may determine an average of the individual predictions P_1 to P_K and compare each individual prediction P_1 to P_K to the average of all predictions P. Predictions P that are significantly higher or lower than the average are deemed noisy and are adjusted to reduce the impact of noise, while consistent values are maintained or enhanced. For example, predictions P higher than a threshold of the average or lower than a threshold of the average may be deemed noisy. This approach helps to smooth out unreliable variations and improve the accuracy and consistency of the final aggregated outcome.

For example, referring to FIG. 4, the PIR module 320 may determine noise across the predictions P_1 to P_4. The PIR module 320 may determine the noise according to any known method. The PIR module 320 may remove noise from each of the predictions P_1 to P_K leaving only the portion of the respective prediction P that is more aligned with the average of all predictions P_1 to P_K. For example, the PIR module 320 may remove portions of the respective prediction P that is higher or lower than the threshold. The PIR module 320 may remove the noise from the predictions P according to any known method.

For example, for a given sample input image, each local expert model LE produces a prediction P∈ [symbol] , where N is a number of classes, h is the image height, and w is the image width. For example, as used herein, a number of classes may refer to a number of possible predictions (e.g., a yes/no prediction would include N=2 classes, a prediction based on a retinal image to detect multiple types of eye disease would include N=the number of types of eye disease, etc.).

If all predictions P_1 to P_K are passed through the SAM 350 (described in more detail later) in a static arrangement during the personalization process, then the SAM 350 will tend to focus only on the prediction of the corresponding local expert model LE. For example, in an example in which the P-MOLE model 300 is trained at a terminal device 20_2 (e.g., at a second medical site, etc.) using local expert models LE from terminal devices 20_1, 20_3, and 20_4 (e.g., at first, third, and fourth medical sites, etc.), the corresponding local expert model LE would be the second local expert model LE_2 corresponding with the terminal device 20_2. In this case, the SAM 350 may mimic the prediction P of the corresponding local expert model LE as the final prediction, even though other predictions P may have more information relevant to the ground truth, and performance of the P-MoLE model 300 may therefore be limited.

According to some example embodiments, to reduce or eliminate this issue, during training, the PIR module 320 may employ random channel shuffling (e.g., reordering of the predictions P_1 to P_K). For example, the PIR module 320 may employ the random channel shuffling with a 10% likelihood before determining the noise across the predictions P_1 to P_K. However, example embodiments are not limited to this example. The random shuffling is not included during prediction by the P-MoLE 300.

According to some example embodiments, the PIR module 320 may denoise each prediction P_1 to P_K in relation to the combined set of predictions P in a pixel-wise manner. For example, the PIR module 320 may remove the noise (e.g., prediction inconsistency) using a rectified linear unit (ReLU). However, example embodiments are not limited to this example.

The PIR module 320 may determine the prediction inconsistency and denoise the prediction to generate a refined prediction P′ according to Equation 1, where an i^{th} pixel of P is denoted by P (i), an i^{th} pixel of P′ is denoted by P′(i), and a mean of i^{th} pixels across all predictions P_1 to P_K is

$$mean ( i ) = \frac{1}{K} {\sum }_{k = 1}^{K} {P}_{k} ( i ) .$$

$${P}^{′} = \{ \begin{matrix}ReLU ( mean ( i ) ) , & if P ( i ) \geq mean ( i ) \\ ReLU ( 2 P ( i ) - mean ( i ) ) , & if P ( i ) < mean ( i )\end{matrix} \qquad Equation 1$$

The ReLU may introduce nonlinearity in the predictions P and turn all negative values to zeros. Larger values, in comparison to the mean from a single prediction P, are likely outliers (e.g., false positives) and are adjusted by using the mean value instead. On the other hand, smaller values, when compared to the mean, are likely false negatives and are adjusted by scaling with the mean at the specific pixel. As a result, noisy pixel predictions are dampened based on the mean of all predictions P from the local expert models LEs.

Improved qualitative results may be obtained by being more aggressive in removing noise. For example, this approach may effectively remove noisy regions in the predictions P. The refined predictions P′ may be concatenated sequentially from 1 to K, resulting in a combined prediction map P_{c}∈ [symbol] .

Since the individual local expert models LE are trained on their respective local data, they may not accurately represent data from other sites. If the testing samples differ significantly from the training samples due to data heterogeneity across multiple sites, the local expert models LE can fail to produce informative predictions, meaning garbage or nearly blank predictions P. Passing these predictions P without any useful information may directly negatively impact the performance of the P-MOLE model 300.

According to some example embodiments, the PIR module 320 may use a “fill missing with the mean” technique during a prediction mode (e.g., after training is complete). For example, the PIR module 320 may replace a failed or blank prediction P with the mean of the other informative predictions P. For example, a prediction P may be determined failed or blank if it contains less than 0.5% positive pixels in its segmentation. However, example embodiments are not limited to this example.

Returning to FIG. 3, Each channel C∈K×N of the combined prediction maps P_{c} contains the refined prediction P′ of each local expert model LE_1 to LE_K. Even after refining the prediction P, some of the refined predictions P′ lack useful information relevant to the ground truth. The PMW module 340 may quantify the usefulness of a channel C from two perspectives: channel importance and informativeness. FIG. 5 shows an example algorithm for generating a weight of a combined prediction map P_{c}.

For the channel importance, the local expert models LE_1 to LE_K may not be equally confident in making a prediction P on a specific sample input image IN. According to some example embodiments, the LC module 330 may determine an importance of each channel C of the predictions P_1 to P_K uniquely according to a learned confidence of each local expert model LE of the plurality of local expert models LE_1 to LE_K. For example, the LC module 330 may determine the confidence automatically on a per-input sample basis by using a linear module supervised with a separate optimizer.

According to some example embodiment, the LC module 330 may generate a confidence probability distribution ρ of each local expert model LE_1 to LE_K for a given input sample by passing the combined prediction map P_{c} through a 1×1 convolutional layer followed by a fully connected linear layer, which may return a confidence vector of length K. The returned confidence vector may then be passed through a softmax layer to return the importance as a probability distribution ρ whose summation equals 1. However, example embodiments are not limited to this example and the confidence probability distribution may be determined according to any known method. The probability distribution ρ is then output to the PMW module 340. The probability distribution ρ may be referred to as weights W of the predictions P_1 to P_K. According to some example embodiments, the weights W may indicate a learned confidence of each local model for the input sample.

According to some example embodiments, the PMW module 340 may generate the informativeness of each refined prediction P′ based on an average pooling and a maximum pooling across the channel which returns the overall summary of information presented in each channel C. The pooling is informative of how much information is present in each channel and the amount of emphasis the model should place on it. Pooling may refer to an operation to calculate a value based on a selection criterion. For example, an average pooling of P_1 corresponds with an average of all values (e.g. pixel values) in P_1. For example, maximum pooling of P_1 corresponds with a maximum value (e.g. pixel value) of all values in P_1.

The PMW module 340 may project the combined prediction map P_{c} using a 1×1 convolutional layer and utilize the projected predictions to compute an average pooling and a maximum pooling of the combined prediction map P_{c}, resulting in Avg ∈ [symbol] and Max ∈ [symbol] . However, example embodiments are not limited to this example and the average and/or maximum pooling may be determined according to any known method. These computed average and maximum pooling values are concatenated with the computed channel confidence ρ separately.

Both of the concatenated average and maximum pooling are passed through a convolution block consisting of a 1×1 convolutional layer followed by a ReLU. Then, the projected average and maximum pooling features are summed together and passed through a sigmoid layer, which generates channel weight CW that captures both channel importance and informativeness. However, example embodiments are not limited to this example and the channel weights may be generated based on the concatenated average and maximum pooling according to any known method.

Returning to FIG. 3, the PMW module 340 may weight each channel C of the prediction map P_{c} based on the channel weights CW. For example, the PMW module 340 may weight each channel C through point-wise multiplication.

According to some example embodiments, predictions P_1 to P_K including the model-learned confidence concatenated with the pooling values summed together after projections, as discussed above, may deliver more useful insights regarding each channel C and may reduce and/or prevent the P-MoLE model 300 from over-fitting.

The output of the PMW module 340, the channel-wise weighted prediction map P′_{c}, is passed through the SAM for dynamic segmentation aggregation.

The SAM module 350 combines the weighted and refined predictions P′_{c} output from the PMW module 340 into a single, cohesive output prediction Q. For example, the prediction Q may be a prediction of at least one malady (e.g., a disease, pathology, degeneration, etc.) based on the medical image or the medical image dataset. According to some example embodiments, the output prediction Q may include a segmentation map. For example, the segmentation map may have dimensions equal to the input image width w×the input image width w. According to example embodiments, the output prediction Q may benefit from the strengths of each individual prediction P_1 to P_K, leading to a more accurate and reliable result.

The improved and weighted predictions P′_{c} provide important insights into which predictions P_1 to P_K are most relevant and deserve more focus. To make the best use of this information, the SAM module 350 operates like a team of experts, each bringing their unique perspectives based on different data sources. The SAM module 350 uses an encoder/decoder process, where it takes all the individual refined and weighted predictions as input and combines them into one final, unified prediction Q. This approach ensures that the final output benefits from the strengths of each individual prediction P_1 to P_K, leading to a more accurate and reliable result.

For example, the SAM module 350 may include a ResNet18-backend Unet model with two encoder and decoder blocks. However, example embodiments are not limited to this example.

The SAM module 350 may translate the weighted and refined predictions P′_{c} into a feature map (e.g., a decoded feature map). For example, the SAM module 350 may translate the weighted and refined predictions P′_{c} using the ResNet18-backend Unet model.

The SAM module 350 may pass the feature map through a 1×1 convolutional layer for projection into an N-class prediction map. The SAM module 350 may then apply sigmoid to generate a final prediction as a segmentation map Q (e.g., an image segmentation map). The segmentation map Q may be referred to as a unified prediction Q.

For example, the SAM module 350 may input P′_{c}∈ [symbol] from the PMW module 340 and output segmentation Q∈ [symbol] .

According to example embodiments, since the local expert models LE_1 to LE-K are frozen throughout and only the P-MoLE-specific parameters (e.g., the LC module 330, the PMW module 34, and/or the SAM module 350) are trained, the P-MoLE model 300 according to example embodiments does not involve a large cost and can scale to large numbers of local expert models LE with little additional overhead. Hence, the scalability of the P-MoLE model 300 according to example embodiments may not present a significant problem. Further, if a new member wishes to contribute, their local model may be easily added to the local expert models LE, whereas in traditional PFL/FL, this flexibility to new members may not exist. In addition, a same architecture may be used for all local expert models LE for simplicity, sharing the weights instead of loading the architecture code, which may reduce the computational overhead.

FIG. 6 is a flowchart illustrating a method for training a P-MoLE model according to example embodiments. The method shown in FIG. 6 may be performed at the server 10 and/or at a terminal device 20. For example, the method shown in FIG. 6 may be performed by the processor 12 of the server 10 and/or the processor 12 of a terminal device 20.

Referring to FIG. 6, at S600 the processor 12 receives a plurality of local expert models LE_1 to LE_K. For example, in an example embodiment where the P-MoLE model 300 is trained at the server 10, the server 10 may receive trained local expert models LE_1 to LE_K from each of terminal devices 20_1 to 20_K respectively.

According to some example embodiments, the P-MoLE model 300 may be trained at a terminal device 20 of the plurality of terminal devices 20_1 to 20_K. In this example, the server 10 may transmit to a terminal device 20 the local expert models LE_1 to LE_K previously received from the plurality of terminal devices 20_1 to 20_K. For example, the server 10 may transmit the plurality of local expert models LE_1 to LE_K to a terminal device 20 in response to a request from the terminal device 20.

The plurality of local expert models LE_1 to LE_K may be frozen. For example, the plurality of local expert models LE_1 to LE_K may no longer be trained after they are transmitted to the server 10. The processor 12 may store the plurality of local expert models LE_1 To LE_K. For example, the processor 12 may store the plurality of local expert models LE_1 To LE_K in the memory 14. For example, the processor 12 may store the plurality of local expert models LE_1 To LE_K as the ToE module 310.

Returning to FIG. 6, at S610, the processor 12 generates a plurality of predictions P_1 to P_K based on an input sample. For example, the ToE module 310 may generate the plurality of predictions P_1 to P_K by inputting the input sample to each of the plurality of local expert models LE_1 to LE_K. Each of the local expert models LE_1 to LE_K may output a prediction P_1 to P_K, respectively.

At S620, the processor 12 performs a random channel shuffling of the predictions P_1 to P_K. For example, the PIR module 320 may employ the random channel shuffling with a 10% likelihood.

At S630, the processor 12 determines a noise of the plurality of predictions P_1 to P_K. For example, the PIR module 320 may determine noise across the predictions P_1 to P_K.

At S640, the processor 12 denoises the plurality of predictions P_1 to P_K. For example, the PIR module 320 may remove noise from each of the predictions P_1 to P_K leaving only the portion of the respective prediction P that is more aligned with the average of all predictions P_1 to P_K. The PIR module 320 may output a plurality of refined (e.g., denoised) predictions P′_1 to P′ K.

At S650, the processor 12 determines an importance of the refined predictions P′_1 to P′ K. For example, The LC module 330 may determine weights W of the channels C of the plurality of refined predictions P′_1 to P′_K. For example, the LC module 330 may generate a confidence probability distribution ρ of each local expert model LE_1 to LE_K for a given input sample.

At S655, the processor 12 determines an informativeness of the refined predictions P′_1 to P′_K. For example, the PMW module 340 may determine the informativeness of each refined prediction P′ 1 to P′ K based on an average pooling and a maximum pooling across the channel.

The LC module 330 and/or the PMW module 340 may be trained during the training of the P-MoLE model 300. For example, the convolutional layers, linear layers, softmax layers, sigmoid layers, and/or the ReLU included in the PMW module 340 may be trained.

At S660, the processor 12 weights each refined prediction P′_1 to P′_K. For example, the PMW module 340 may weight each channel C of the refined predictions P′_1 to P′ K based on based on a confidence level (e.g., an importance and/or an informativeness) of the respective refined prediction P′_1 to P′_K. For example, the PMW module 340 may weight each channel C based on the probability distribution ρ and/or the average pooling and a maximum pooling. For example, the PMW module 340 may weight each channel C through point-wise multiplication. The PMW module 340 may output the weighted and refined predictions as a prediction map P′_{c}.

At S670, the processor 12 may determine an output prediction Q based on the weighted refined predictions P′_1 to P′ K. For example, the SAM module 350 may combine the weighted and refined predictions P′_{c} output from the PMW module 340 into a single, cohesive output prediction Q.

The SAM module 350 may be trained during the training of the P-MoLE model 300. For example, the ResNet18-backend Unet model, convolutional layers, and/or sigmoid layers included in the SAM module 350 may be trained.

At S680, the processor 12 outputs the trained P-MoLE model 300. For example, in an example where the training is performed at the server 10, the server 10 may transmit the trained P-MOLE model 300 to the requesting terminal device 20. The terminal device 20 may store the trained P-MoLE model 300 in the memory 14. The trained P-MoLE model 300 may include the plurality of local expert models LE_1 to LE_K, the ToE module 310, the PIR module 320, the trained LC module 330, the trained PMW module 340, and/or the trained SAM module 350.

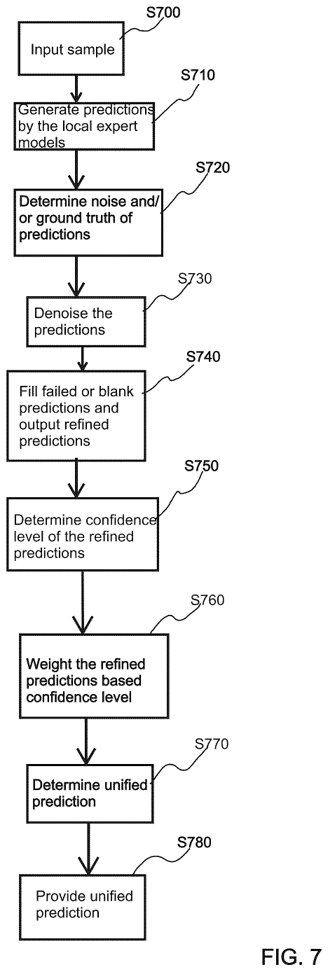

FIG. 7 is a flowchart illustrating a method for generating a prediction using a P-MoLE model according to example embodiments. The method shown in FIG. 7 may be performed at the server 10 and/or at a terminal device 20. For example, the method shown in FIG. 7 may be performed by the processor 12 of the server 10 and/or the processor 12 of a terminal device 20.

Referring to FIG. 7, at S700 the processor 12 inputs an input sample. For example, the input sample may be an image or a plurality of images (e.g., an image dataset). The processor 12 may input the input sample to the P-MoLE model 300. For example, the P-MoLE model 300 may be a trained P-MOLE model 300.

At S710, the processor 12 generates a plurality of predictions P_1 to P_K based on an input sample. For example, the ToE module 310 may generate the plurality of predictions P_1 to P_K by inputting the input sample to each of the plurality of local expert models LE_1 to LE_K. Each of the local expert models LE_1 to LE_K may output a prediction P_1 to P_K, respectively.

At S720, the processor 12 determines noise of the plurality of predictions P_1 to P_K. For example, the PIR module 320 may determine noise across the predictions P_1 to P_K.

At S730, the processor 12 denoises the plurality of predictions P_1 to P_K. For example, the PIR module 320 may remove noise from each of the predictions P_1 to P_K.

At S740, the processor 12 fills failed or blank predictions. For example, the PIR module 320 may use a “fill missing with the mean” technique. For example, the PIR module 320 may replace a failed or blank prediction P with the mean of the other informative predictions P. For example, a prediction P may be determined failed or blank if it contains less than 0.5% positive pixels in its segmentation. However, example embodiments are not limited to this example.

The PIR module 320 may output a plurality of refined (e.g., denoised and/or replaced) predictions P′_1 to P′ K.

At S750, the processor 12 determines a confidence level (e.g., an importance and/or informativeness) of the refined predictions P′_1 to P′_K. For example, the LC module 330 may determine the importance of the refined predictions P′_1 to P′ K. The PMW module 340 may determine the informativeness of the refined predictions P′_1 to P′ K and concatenate the importance with the informativeness.

At S760, the processor 12 weights each refined prediction P′_1 to P′_K. For example, the PMW module 340 may weight each channel C of the refined predictions P′_1 to P′ K based on the concatenated importance and informativeness. For example, the PMW module 350 may weight each channel C through point-wise multiplication. The PMW module 350 may output the weighted and refined predictions as a prediction map P′_{c}.

At S770, the processor 12 may determine an output prediction Q based on the weighted refined predictions P′_1 to P′_K. For example, the SAM module 350 may combine the weighted and refined predictions P′_{c} output from the PMW module 340 into a single, cohesive output prediction Q.

At S780, the processor 12 may provide the output prediction Q. For example, the output prediction Q may be displayed on the user interface 18 and/or the output prediction Q may be stored in the memory 14.

Although the terms first, second, etc. may be used herein to describe various elements, these elements should not be limited by these terms. These terms are only used to distinguish one element from another. For example, a first element could be termed a second element, and similarly, a second element could be termed a first element, without departing from the scope of this disclosure. As used herein, the term “and/or,” includes any and all combinations of one or more of the associated listed items.

When an element is referred to as being “connected,” or “coupled,” to another element, it can be directly connected or coupled to the other element or intervening elements may be present. By contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting. As used herein, the singular forms “a,” “an,” and “the,” are intended to include the plural forms as well, unless the context clearly indicates otherwise. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Specific details are provided in the following description to provide a thorough understanding of example embodiments. However, it will be understood by one of ordinary skill in the art that example embodiments may be practiced without these specific details. For example, systems may be shown in block diagrams so as not to obscure the example embodiments in unnecessary detail. In other instances, well-known processes, structures and techniques may be shown without unnecessary detail in order to avoid obscuring example embodiments.

As discussed herein, illustrative embodiments will be described with reference to acts and symbolic representations of operations (e.g., in the form of flow charts, flow diagrams, data flow diagrams, structure diagrams, block diagrams, etc.) that may be implemented as program modules or functional processes include routines, programs, objects, components, data structures, etc., that perform particular tasks or implement particular abstract data types and may be implemented using existing hardware at, for example, existing network apparatuses, elements or entities including cloud-based data centers, computers, cloud-based servers, or the like. Such existing hardware may be processing or control circuitry such as, but not limited to, one or more processors, one or more Central Processing Units (CPUs), one or more controllers, one or more arithmetic logic units (ALUs), one or more digital signal processors (DSPs), one or more microcomputers, one or more field programmable gate arrays (FPGAs), one or more System-on-Chips (SoCs), one or more programmable logic units (PLUS), one or more microprocessors, one or more Application Specific Integrated Circuits (ASICs), or any other device or devices capable of responding to and executing instructions in a defined manner.

Although a flow chart may describe the operations as a sequential process, many of the operations may be performed in parallel, concurrently or simultaneously. In addition, the order of the operations may be re-arranged. A process may be terminated when its operations are completed, but may also have additional steps not included in the figure. A process may correspond to a method, function, procedure, subroutine, subprogram, etc. When a process corresponds to a function, its termination may correspond to a return of the function to the calling function or the main function.

As disclosed herein, the term “storage medium,” “computer readable storage medium” or “non-transitory computer readable storage medium” may represent one or more devices for storing data, including read only memory (ROM), random access memory (RAM), magnetic RAM, core memory, magnetic disk storage mediums, optical storage mediums, flash memory devices and/or other tangible machine-readable mediums for storing information. The term “computer-readable medium” may include, but is not limited to, portable or fixed storage devices, optical storage devices, and various other mediums capable of storing, containing or carrying instruction(s) and/or data.

Furthermore, example embodiments may be implemented by hardware, software, firmware, middleware, microcode, hardware description languages, or any combination thereof. When implemented in software, firmware, middleware or microcode, the program code or code segments to perform the necessary tasks may be stored in a machine or computer readable medium such as a computer readable storage medium. When implemented in software, a processor or processors will perform the necessary tasks. For example, as mentioned above, according to one or more example embodiments, at least one memory may include or store computer program code, and the at least one memory and the computer program code may be configured to, with at least one processor, cause a network apparatus, network element or network device to perform the necessary tasks. Additionally, the processor, memory and example algorithms, encoded as computer program code, serve as means for providing or causing performance of operations discussed herein.

A code segment of computer program code may represent a procedure, function, subprogram, program, routine, subroutine, module, software package, class, or any combination of instructions, data structures or program statements. A code segment may be coupled to another code segment or a hardware circuit by passing and/or receiving information, data, arguments, parameters or memory contents. Information, arguments, parameters, data, etc. may be passed, forwarded, or transmitted via any suitable technique including memory sharing, message passing, token passing, network transmission, etc.

The terms “including” and/or “having,” as used herein, are defined as comprising (i.e., open language). The term “coupled,” as used herein, is defined as connected, although not necessarily directly, and not necessarily mechanically. Terminology derived from the word “indicating” (e.g., “indicates” and “indication”) is intended to encompass all the various techniques available for communicating or referencing the object/information being indicated. Some, but not all, examples of techniques available for communicating or referencing the object/information being indicated include the conveyance of the object/information being indicated, the conveyance of an identifier of the object/information being indicated, the conveyance of information used to generate the object/information being indicated, the conveyance of some part or portion of the object/information being indicated, the conveyance of some derivation of the object/information being indicated, and the conveyance of some symbol representing the object/information being indicated.

According to example embodiments, network apparatuses, elements or entities including cloud-based data centers, computers, cloud-based servers, or the like, may be (or include) hardware, firmware, hardware executing software or any combination thereof. Such hardware may include processing or control circuitry such as, but not limited to, one or more processors, one or more CPUs, one or more controllers, one or more ALUs, one or more DSPs, one or more microcomputers, one or more FPGAs, one or more SoCs, one or more PLUS, one or more microprocessors, one or more ASICs, or any other device or devices capable of responding to and executing instructions in a defined manner.

What is claimed is:

1. A method to generate a unified prediction, the method comprising:
generating a plurality of predictions of an input based on a plurality of local prediction models, wherein the plurality of local prediction models are trained for image segmentation;
determining a confidence level of the plurality of predictions, wherein the determining the confidence level includes determining an importance of each prediction based on an average pooling of the prediction and a maximum pooling of the prediction;
assigning weights to the plurality of predictions based on the respective confidence levels; and
generating the unified prediction based on the plurality of predictions and the respective weights, wherein the generating the unified prediction includes generating an image segmentation of the input.

2. The method of claim 1, wherein the input is at least one of a medical image or a medical image dataset, and
wherein the generating the unified prediction includes generating a prediction of at least one malady based on the medical image or the medical image dataset.

3. The method of claim 1, wherein the input is at least one of a medical image or a medical image dataset, and
the generating the unified prediction includes generating an image segmentation map.

4. The method of claim 1, wherein each prediction model of the plurality of prediction models corresponds with a different respective source of a plurality of sources.

5. The method of claim 4, wherein each local prediction model is trained by the respective different source.

6. The method of claim 4, wherein the method further comprises:
receiving the plurality of local prediction models from the different respective sources.

7. The method of claim 1, further comprising:
denoising the plurality of predictions by adjusting predictions differing from an average of the plurality of predictions by greater than a threshold.

8. The method of claim 7, wherein the denoising includes replacing a failed prediction with the average of the plurality of predictions.

9. The method of claim 1, further comprising:
training a general prediction model by the determining, the assigning, and the generating.

10. The method of claim 9, wherein the general prediction model includes the plurality of local prediction models.

11. The method of claim 1, wherein the generating the unified prediction includes generating the unified prediction using a convolutional network based on the plurality of predictions and the respective weights.

12. An apparatus comprising:
at least one memory storing instructions; and
at least one processor configured to execute the instructions and cause the apparatus to
generate a plurality of predictions of an input based on a plurality of local prediction models, wherein the plurality of local prediction models are trained for image segmentation,
determine a confidence level of the plurality of predictions, wherein the determining the confidence level includes determining an importance of each prediction based on an average pooling of the prediction and a maximum pooling of the prediction,
assign weights to the plurality of predictions based on the respective confidence levels, and
generate a unified prediction based on the plurality of predictions and the respective weights, wherein the generating the unified prediction includes generating an image segmentation of the input.

13. The apparatus of claim 12, wherein the input is at least one of a medical image or a medical image dataset, and
wherein generate the unified prediction includes generating a prediction of at least one malady based on the medical image or the medical image dataset.

14. The method of claim 12, wherein the input is at least one of a medical image or a medical image dataset, and
generate the unified prediction includes generating an image segmentation map.

15. The apparatus of claim 12, wherein each prediction model of the plurality of prediction models corresponds with a different respective source of a plurality of sources.

16. The method of claim 15, wherein each local prediction model is trained by the respective different source.

17. The apparatus of claim 15, wherein the apparatus is further caused to:
receive the plurality of local prediction models from the different respective sources.

18. The apparatus of claim 12, wherein the apparatus is further caused to:
denoise the plurality of predictions by adjusting predictions differing from an average of the plurality of predictions by greater than a threshold.

19. The method of claim 18, wherein the denoising includes replacing a failed prediction with the average of the plurality of predictions.

20. A non-transitory computer-readable medium, storing instructions for performing:
generating a plurality of predictions of an input based on a plurality of local prediction models, wherein the plurality of local prediction models are trained for image segmentation;
determining a confidence level of the plurality of predictions, wherein the determining the confidence level includes determining an importance of each prediction based on an average pooling of the prediction and a maximum pooling of the prediction;
assigning weights to the plurality of predictions based on the respective confidence levels; and
generating the unified prediction based on the plurality of predictions and the respective weights, wherein the generating the unified prediction includes generating an image segmentation of the input.
